(19) United States
(12) Patent Application Publication
(10) Pub. No.: US 2023/0107605 A1
(43) Pub. Date: Apr. 6, 2023

(54) CARTRIDGE WITH NICOTINE

(71) Applicant: McNeil AB, Helsingborg (SE)

(72) Inventor: Corrado Tasselli, Malmö (SE)

(21) Appl. No.: 17/907,108

(22) PCT Filed: Mar. 26, 2021
(86) PCT No.: PCT/EP2021/057955

§ 371 Date: Sep. 23, 2022

(30) Foreign Application Priority Data
Mar. 27, 2020 (SE) 2050337-1

(51) Int. Cl. A24F 40/42; A24F 47/00; A24F 40/10; A24B 15/167; A24F 40/44; A24F 40/48; A24F 7/00; A24F 40/53

(52) U.S. Cl. CPC A24F 40/42; A24F 47/00; A24F 40/10; A24B 15/167; A24F 40/44; A24F 40/48; A24F 7/00; A24F 40/53

(57) ABSTRACT

The invention relates to a nicotine containing cartridge assembly for use in a medical electronic nicotine delivery device, having a prolonged life time that remains over the treatment period, wherein the treatment is to help a person stop smoking.

### FIELD OF INVENTION

The invention relates to a nicotine containing cartridge assembly for use in a medical electronic nicotine delivery device, having a prolonged life time that remains over the treatment period, wherein the treatment is to help a person stop smoking.

### BACKGROUND OF INVENTION

During the last years electronic delivery systems—devices for the delivery of nicotine in a liquid have been developed and still continue to be developed. They are mainly developed by the tobacco industries to provide smoking systems.

However, there are still only one medical device for the delivery of nicotine out on the market that help people to quit smoking. That medical device gives a fast craving release and is sold under the trademark Nicorette™ QuickMist™ which is a mouth spray to be applied to the oromucosa from which the nicotine compound is readily absorbed into the blood stream to give a fast craving release. The nicotine liquid formulation in that product is disclosed in WO2007133140. Behind the trademark Nicorette are a number of differentiated medical products, including patches, gums, lozenges and sprays wherein all are developed and introduced onto the market to help people stop smoking. If the product is a medical device, it needs to be developed to be safe and fulfil the medical device regulations in the different countries the medical device is developed for.

Nicotine in the free form is a very nasty chemical compound, being very toxic, volatile and corrosive to many materials. Thus, all the different components within a device as well as in other parts that comes into contact, with the nicotine composition, needs to be resistant against nicotine. No degradation products are allowed, to be produced that might be toxic for the human using the medical device. The nicotine composition used in a medical device should not contain components or produce any unhealthy compounds, during use of the regulated medical device, which is different compared to the e-cigarettes which are not regulated.

WO2019154811 discloses a cartridge assembly utilized in combination with an electronic nicotine delivery system delivering a controlled dose of nicotine to a user on demand. The cartridge assembly includes a reservoir with a material for holding liquid nicotine or a liquid nicotine solution and is constructed from a material that is chemically resistant to nicotine or nicotine solution.

EP3574902 discloses a pharmaceutical formulation comprising nicotine. The formulation can be aerosolised at ambient temperature for delivery via inhalation. It also relates to a method of delivering nicotine to a subject via inhalation, and specifically via the use of a nebuliser.

None of the cited prior art relates to the invention or the same technical problem to be solved.

### SUMMARY OF THE INVENTION

The inventors have been facing the problem of developing a medical electronic nicotine delivery device having a life time which allows the user to quit smoking. Recommendations upon quitting smoking according to some medical doctors is 6 weeks of replacement therapy followed by 6 weeks of tapering down. This means that for a smoker smoking 20 cigarettes a day, the components of the medical device in contact with the nicotine composition in any form should withstand around at least 20 000 cycles of inhalation. One of the components that the medical electronic nicotine delivery device utilises is a wick in contact with the nicotine composition. The wick may be made of a porous material, such as a polymeric porous material, that may be a thermoplastic polyester. It has been found that wicks degrade and/or change during the life cycle of the medical device. The inventors facing this problem of the alteration of the wick during the life time found that by altering the amount of the ingredients in the nicotine composition to a specific range as well as solely having nicotine in the free form, propylene glycol and water present gave rise to an improvement in the life of the wick and it survived more than 20 000 cycles. In addition, the inventors found that the nicotine composition specified herein advantageously does not affect other components of the device, such as the fibrous material which may be present in the reservoir.

The present invention provides a nicotine containing cartridge assembly (1) for use in a medical electronic nicotine delivery device (2) comprising:
a body assembly (3) having an interior surface defining an interior volume, the interior volume including a reservoir (4), wherein the reservoir has an interior volume, wherein the interior volume (8) of the reservoir (4) contains a composition comprising, consisting essentially of or consisting of:
a. nicotine or a salt thereof in an amount of from about 2% w/w to about 6% w/w,
b. propylene glycol in an amount of from 50% w/w to about 65% w/w; and
c. water in an amount of from about 35% w/w to about 50% w/w.

Such a composition within a cartridge assembly has been found to be particularly compatible and not cause significant degradation during the use of the cartridge.

The reservoir may be configured to receive a wick of the medical electronic nicotine delivery device such that the wick contacts the composition. In this arrangement the compatibility of the composition with wick materials is particularly beneficial.

The reservoir may comprise a port to allow the insertion of the wick therethrough. The port is an opening allowing the wick to enter the reservoir and contact the composition contained within. When the reservoir has a top wall, bottom wall and side walls, the port may be in the bottom wall of the reservoir.

As described herein, the wick that can be used with the cartridge assembly may be formed from a range of suitable materials. The invention has been found to be particularly effective with porous polymeric materials, such as porous thermoplastic polymeric materials. Such polymeric materials include polyesters, in particular thermoplastic polyesters. Useful thermoplastic polyesters include polyethylene terephthalate (PET) and polybutylene terephthalate (PBT). The wick may consist of only one material, such as the polymeric material. This reduces the risk of producing contaminants during use of the wick. The wick may be a rigid wick that has sufficient rigidity to maintain its own shape, without external support, when inserted into the reservoir, especially over the course of repeated insertions.

The wick may be porous due to being formed from fibrous material. In other words, the wick comprises a plurality of fibres, such as polymeric fibres. These can be compacted together to form the wick. This approach has been found particularly effective at producing a wick with the required rigidity and without utilizing further, possibly contaminating, materials.

The wick may be subjected to heat by the medical electronic nicotine delivery device in order to form an aerosol from the composition. The wick functions to transfer the composition from the reservoir to the heated region of the wick. The heat may be provided to the wick by a heater that surrounds the wick along a portion of its length. The heater may be a heating element encapsulated in an inert material, such as a ceramic.

The interior volume of the reservoir may comprise a fibrous material or a sponge, wherein the fibrous material or sponge contains the composition. This is a convenient way of retaining the composition within the reservoir until contacted by the wick during use. Such an arrangement benefits from the compatibility of the present compositions with fibrous materials and sponges. The fibrous material or sponge may be saturated with the composition. A fibrous material is one that comprises a plurality of fibres. A sponge is a continuous matrix of material with an open porosity that can retain the composition.

Particularly effective fibrous materials and sponges have been found to comprise polymeric materials, such as thermoplastic polymeric materials. Such polymeric materials include polyesters, in particular thermoplastic polyesters. Useful thermoplastic polyesters include polyethylene terephthalate (PET) and polybutylene terephthalate (PBT). The fibrous material or sponge in the reservoir may consist of only one material, such as the polymeric material. This reduces the risk of producing contaminants within the composition.

The present invention also provides a nicotine containing cartridge assembly (1) for use in a medical electronic nicotine delivery device (2) comprising:
a) a body assembly (3) having an interior surface defining an interior volume, the interior volume including, a reservoir (4), the reservoir (4) having side walls (7) having an inner surface and an outer surface, the inner surface of side walls (7) defining an interior volume (8) of the reservoir (4); wherein the reservoir (4) contains a composition comprising, consisting essentially of or consisting of:
   a) nicotine or a salt thereof in an amount of from about 2% w/w to about 6% w/w,
   b) propylene glycol in an amount of from 50% w/w to about 65% w/w and
   c) water in an amount of from about 35% w/w to about 50% w/w,
b) two conduits (12) formed between the outer surface of side walls (7) of the reservoir (4) and the interior surface of the body assembly (3), the conduits (12) having open top ends (13), and open bottom ends (14), and being configured for conducting aerosolized nicotine; and
c) a manifold (15) for holding and distributing aerosolized nicotine, the manifold (15) positioned above a top wall (5) of the reservoir (4), the manifold (15) having an open top end (16) and two open bottom ends (17), and two interior channels (18) connecting the open top end (16) with the open bottom ends (17), wherein the open bottom ends (17) are secured onto the open top ends (13) of the conduits (12) and are in fluid communication with the conduits (12).

By the use of such a specific nicotine composition in the reservoir it is possible to enhance the compatibility of the nicotine composition with the other components of the medical electronic nicotine delivery system. For example, the specific nicotine composition in the reservoir can maintain the quality of the wick during the expected life time being at least 20 000 cycles. It also has a good compatibility with materials that may be used in the reservoir.

The present invention also provides a kit comprising the nicotine containing cartridge assembly as described herein as well as a medical electronic nicotine delivery device as described herein.

The present invention also provides a kit comprising at least one nicotine containing cartridge assembly as described herein (e.g. at least two nicotine containing cartridge assemblies).

The present invention also provides a medical electronic nicotine delivery system comprising the medical electronic nicotine delivery device described herein and comprising a cartridge assembly as described herein.

The present invention also provides a medical electronic nicotine delivery device, comprising a housing, a cartridge assembly as described herein, an electrical module, a power supply, a heater, and a vaporization chamber.

The present invention also provides the use of the medical electronic nicotine delivery device with the nicotine containing cartridge assembly as described herein for the treatment of a human having tobacco dependency and suffering from cravings.

The present invention further provides the use of a nicotine-containing composition to reduce degradation of a component of a medical electronic nicotine delivery system during the use of the system, wherein the composition comprises
nicotine or a salt thereof in an amount of from about 2% w/w to about 6% w/w, propylene glycol in an amount of from 50% w/w to about 65% w/w; and water in an amount of from about 35% w/w to about 50% w/w.

The present invention is particularly effective at reducing the degradation of a wick described herein and a fibrous material or sponge within a reservoir described herein.

To be able to introduce onto the market, the device and the cartridge containing the nicotine composition as a medical device all the components as well as the nicotine composition and the chemicals must be made under good manufacturing practices (GMP) to be able to get it registered as a Medical product and thus there are higher demands compared to entering onto the market with for example an E-cigarette.

### BRIEF DESCRIPTION OF THE DRAWINGS

FIG. 1 is a schematic perspective view of a cartridge assembly.

FIG. 2 is a schematic side view of the cartridge assembly of FIG. 1.

FIG. 3 is a schematic side view of the cartridge assembly of FIG. 1.

FIG. 4 is a schematic side view of the cartridge assembly of FIG. 1.

FIG. 5 is a cross-section of the cartridge assembly of FIG. 1.

FIG. 6 is a cross-section of the cartridge assembly of FIG. 1.

FIG. 7 is a second exploded diagrammatic view of the cartridge assembly.

FIG. 8 is a schematic perspective view of the electronic nicotine delivery device for the delivery of nicotine from a cartridge assembly as shown in FIG. 1 to FIG. 6.

FIG. 9 is a diagrammatic representation of the electronic nicotine delivery system.

FIG. 10 is a cross section view of the electronic nicotine delivery system of FIG. 9.

FIG. 11 is a partial cutaway perspective view of the electronic nicotine delivery system of the present invention of FIG. 9.

### DETAILED DESCRIPTION OF EMBODIMENTS OF THE INVENTION

### Definitions

In the context of the present application the following definitions apply:

The calculation of the amount of nicotine present in the composition is calculated as the free base form of nicotine.

The term “% w/w” is intended to mean the percentage of an ingredient(s)/total percentage by weight.

The term “cycle” means the time period that starts from when the user starts inhaling, then the heater in the device starts heating up, and heats the nicotine composition, the nicotine composition becomes vaporized/aerosolized and transferred from the device through the mouth piece into the human being and then the device is turned off. The cycle may take about 30 second to one minute.

### Nicotine Composition in the Reservoir

The nicotine composition present in the reservoir (4) has been developed to fulfil a number of criteria; being completely aerosolized/vaporized to provide a specific amount of nicotine in one cycle as defined above to give craving relief, i.e., 2-6 mg in one cycle, no visible smoke since the medical device should not be associated with a cigarette, no influence on the lifetime of the wick or any other part of the cartridge or device being in contact with the nicotine composition, especially the wick and any fibrous material. In addition no unhealthy compounds, such as fragrances and flavors should be included.

The problems have been solved by developing a nicotine composition comprising, consisting essentially of or consisting of nicotine or a salt thereof in an amount of from about 2% w/w to about 6% w/w, propylene glycol in an amount of from 50% w/w to about 65% w/w and water in an amount of from about 35% w/w to about 50% w/w. The composition should be substantially free from other chemical compounds, such as no other chemical compounds should be present in the composition to reduce the possibility to develop toxic compounds when the composition is heated up.

The amount of nicotine in the composition may vary depending on how addicted the user is and is such as from about 2% w/w to about 6% w/w such as 2% w/w, 3% w/w, 4% w/w, 5% w/w or 6% w/w.

The propylene glycol is present from 50% w/w to 65% w/w, such as 55% w/w to 60% w/w, 56% w/w, 57% w/w, 58% w/w, 59% w/w, 60% w/w, 61% w/w, 62% w/w, 63% w/w, 64% w/w or 65% w/w.

The water is present in an amount from about 35% w/w to about 50% w/w, such as 36% w/w, 37% w/w, 38% w/w, 39% w/w, 40% w/w, 41% w/w, 42% w/w, 43% w/w, 44% w/w, 45% w/w, 46% w/w, 47% w/w, 48% w/w or 50% w/w.

These amounts of nicotine, propylene glycol and water are particularly advantageous, because they give rise to an improvement of the life of the wick, such that it may survive more than 20 000 cycles. This improvement is observed particularly when the wick comprises, consists essentially of or consists of a porous (and optionally rigid) material, such as a porous thermoplastic polyester or another polymer (for example, porous polyethylene terephthalate or PET).

Furthermore, these amounts of nicotine, propylene glycol and water also advantageously preserve a fibrous material which may be present in the reservoir as described hereinbelow. The fibrous material improves the contact between the nicotine composition in the reservoir and the wick. If the fibrous material were not present, then as the nicotine composition in the reservoir became depleted, contact between the nicotine composition and the wick could become more difficult and possibly intermittent.

Examples of nicotine salts include, but are not limited to, formic (2:1), acetic (3:1), propionic (3:1), butyric (3:1), 2-methylbutyric (3:1), 3-methylbutynic (3:1), valeric (3:1), lauric (3:1), palmitic (3:1), tartaric (1:1) and (2:1), citric (2:1), malic (2:1), oxalic (2:1), benzoic (1:1), gentisic (1:1), gallic (1:1), phenylacetic (3:1), salicylic (1:1), phthalic (1:1), picric (2:1), sulfosalicylic (1:1), tannic (1:5), pectic (1:3), alginic (1:2), hydrochloric (2:1), chloroplatinic (1:1), silcotungstic (1:1), pyruvic (2:1), glutamic (1:1), and aspartic (1:1) salts of nicotine. While the use of the free base of nicotine is generally preferred, the use of such salts may be desirable to lower the pH to potentially reduce irritation for liquid formulations containing a high concentration of nicotine.

The nicotine composition is free from glycerol, ethanol, flavor, fragrances or other ingredients to be as safe as possible and not possibility to produce any toxic compounds during the use of the cartridge in the medical device cycles.

### Cartridge

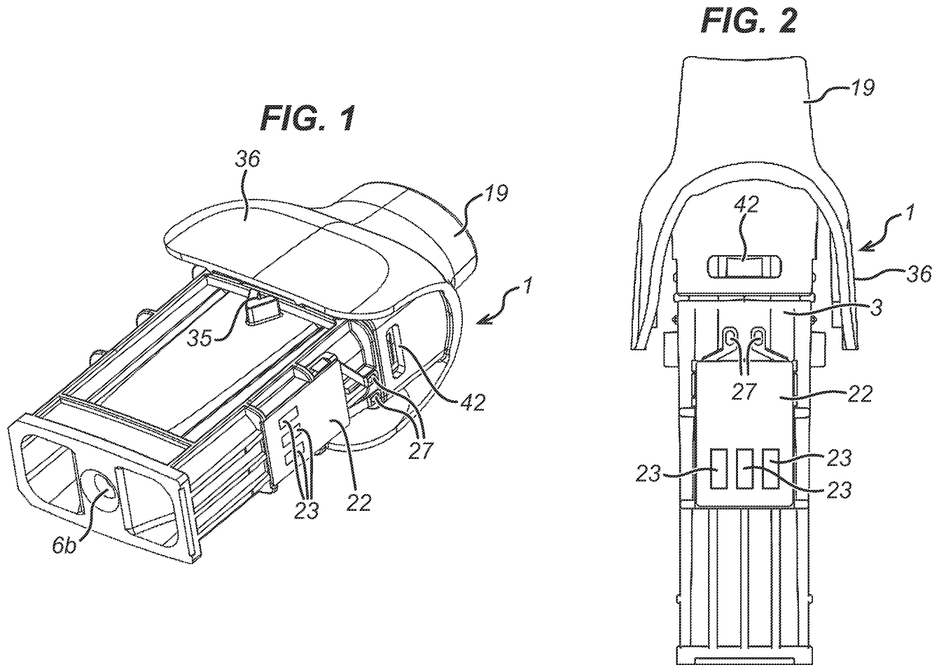

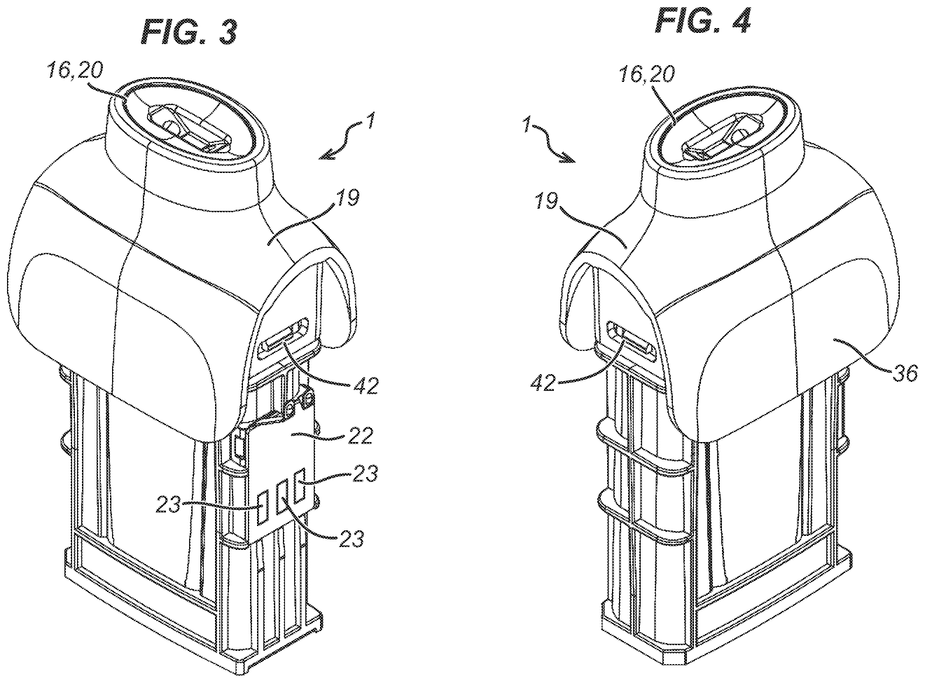

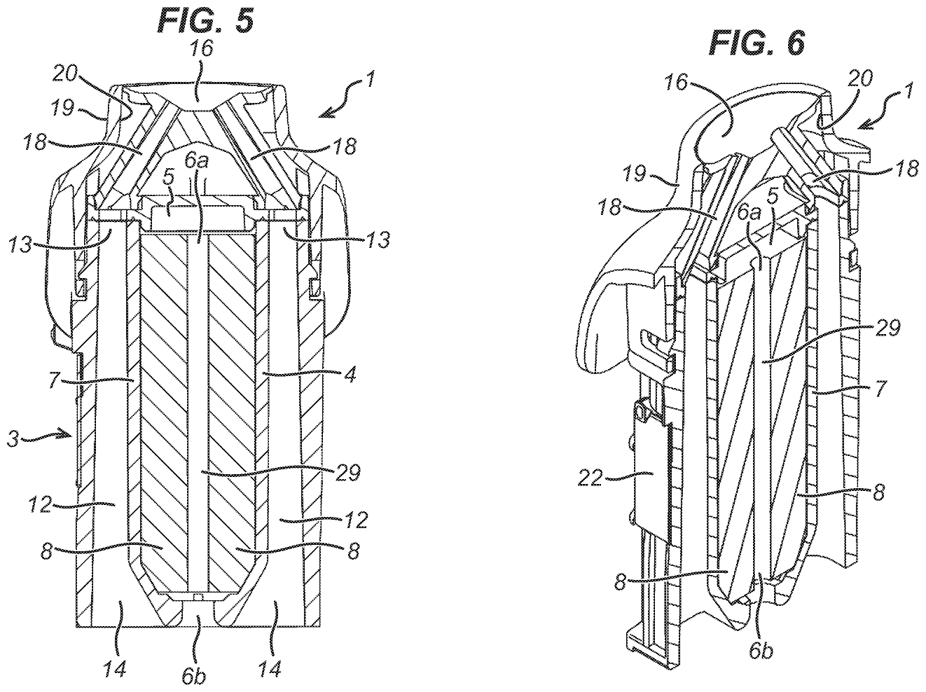

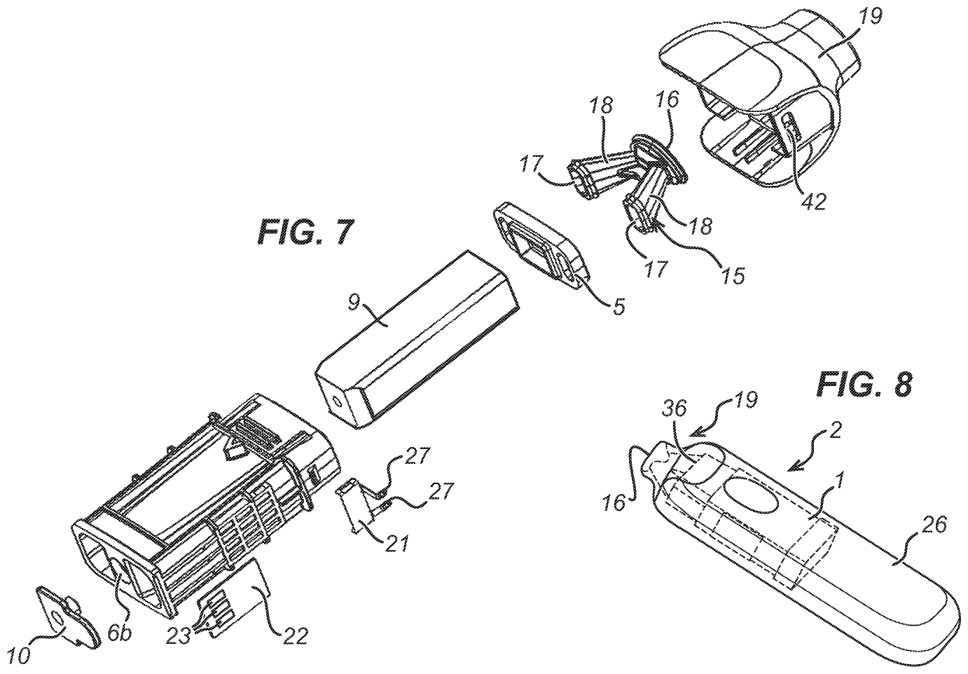

In accordance with the present invention, as illustrated in FIGS. 1-11, a cartridge assembly (1) for a medical electronic nicotine delivery device (2) is disclosed. The cartridge assembly (1) comprises a body assembly (3) with a reservoir (4) configured to hold a nicotine composition. The reservoir (4) may be elongate. The bottom of the cartridge may include a plug (10), described in greater detail below with respect to FIG. 7, which is removably retained in the port or wick opening (29) prior to use and is removed for use.

The cartridge assembly (1) is preferably made of a medical grade, chemical resistant material, such as a thermoplastic material. The cartridge assembly (1) may be made from any suitable nicotine resistant polymeric material or metallic material, including stainless steel. The wick (11) is preferably chemically resistant to nicotine, is temperature resistant, and preferably rigid to prevent collapse during insertion into the reservoir (4) or during use. A more detailed description of the wick (11) is given below. A fibrous material or sponge (9) or any liquid holding element may be arranged in the reservoir (4) and extends to a position close to the first/bottom end of reservoir (4). When the wick (11) has been pushed into the cartridge assembly (1) the fibrous material/sponge (9) can engage the wick (11) so as to allow fluid present in the reservoir (4) to be transported through the wick (11) to the heater (32), preferably an electric heater or heating element, within the housing (26) of the electronic nicotine delivery system (2).

The cartridge assembly (1) as set forth herein is arranged for use in a medical electronic nicotine delivery device (2).

The body assembly (3) comprises, two air-let channels/conduits (12) that extend within the body assembly (3) exterior to and along the reservoir (4).

As set forth above, a single use, tamper resistant cartridge assembly (1) is utilized as an integral component in an electronic nicotine delivery system (2) for smoking cessation. Referring, in greater detail, to FIGS. 1-11, there is illustrated a cartridge assembly (1) in accordance with the present invention. In one exemplary embodiment, the cartridge assembly (1) comprises a body assembly (3) having an interior surface defining an interior volume. The interior volume of the body assembly (3) includes a reservoir (4), the reservoir (4) having top wall (5) with an optional opening (6a) formed therein, a bottom wall, and side walls (7) having an inner surface and an outer surface, the inner surface of the side walls (7) defining an interior volume (8) of the reservoir (4). The interior volume (8) of the reservoir (4) may comprise a nicotine saturated fibrous material (9) or a sponge therein.

The cartridge assembly (1) also includes a port (6b) formed in the bottom wall of the body assembly (3) of the reservoir (4) to allow insertion of a wick (11) from the electronic nicotine delivery device (2). The cartridge assembly (1) further includes two conduits (12) formed between the outer surface of side walls (7) of the reservoir (4) and the interior surface of the body assembly (3), the conduits (12) having open top ends (13) proximate the top wall (5) of the reservoir (4) and open bottom ends (14) proximate the bottom wall of the reservoir (4). The conduits (12) are configured for holding and distributing aerosolized nicotine as is explained in much greater detail subsequently.

The cartridge assembly (1) still further includes a manifold (15) positioned above the top wall (5) of the reservoir (4), the manifold (15) is configured for holding and distributing aerosolized nicotine. In one embodiment, the manifold (15) includes an open top end (16), two open bottom ends (17), and two interior channels (18) connecting the open top end (16) to the open bottom ends (17), wherein the open bottom ends (17) of the manifold (15) being secured onto the open top ends (13) of the conduits (12) of the cartridge assembly (1) so as to form a continuous path from the open top end (16) to the open bottom ends (14) of the conduits (12) for communicating the aerosolized nicotine.

The cartridge assembly (1) still further includes a mouthpiece (19) positioned above the manifold (15), having an outlet (20) in fluid communication with the interior channels (18) of the manifold (15). The mouthpiece (19) being configured for delivering aerosolized nicotine into the mouth of a user. The mouthpiece (19) may be secured to the body assembly (3) via any suitable means, including snap fit features (42) as illustrated in detail in FIGS. 1-4 and 7.

The cartridge assembly (1) further includes at least one of an anti-counterfeit element (22) and an anti-reuse element (21) mounted to the outer surface of the body assembly (3). The anti-counterfeit element (22) is configured to preclude use of an unauthorized cartridge assembly in the electronic nicotine delivery system (2) while the anti-reuse element (21) is configured to preclude reuse of the cartridge assembly (1) once removed from the electronic nicotine delivery system (2) as illustrated in FIGS. 1 and 7.

Referring to FIG. 11, there is illustrated a female mating component (34) in housing (26) of medical electronic nicotine delivery device (2) that interlocks with male component (35), illustrated in FIG. 1, on an outer surface of the body assembly (3) of the cartridge assembly (1). These female/male components may comprise any suitable shape or any suitable mechanisms for locking the cartridge assembly (1) to the electronic nicotine delivery device (2).

In FIG. 7, there is illustrated a plug (10) which covers the open bottom part of the cartridge sealing the reservoir (4), so that the composition held in the reservoir (4), will be maintained within the reservoir (4) and protected from contamination. The plug (10) may be formed from any suitable material that is resistant to nicotine/nicotine composition. The plug (10) is configured to prevent accidental leakage of the nicotine/nicotine composition contained in the reservoir (4).

As set forth above, more particular descriptions of the cartridge assembly (1) and the medical electronic nicotine delivery device (2) is given below as well as a description of the nicotine formulations and operation of the device.

FIGS. 1-11 provide a more detailed illustration of the cartridge assembly (1) and device (2) of the present invention. Referring now to FIGS. 1-11, the cartridge assembly (1) comprises at a first/bottom end including a plug (10) and at a second/top end opposite to the first/bottom end a mouthpiece (19). A reservoir (4) is provided in a body assembly (3) between the first end and the second end. In various exemplary embodiments, the body assembly (3) is elongate and comprises an elongate passage extending from first end of body assembly (3) to second end of body assembly (3). The reservoir (4) is arranged at a central position between conduits/air-let channels (12) forming the elongate passage. The two conduits/air-let channels (12) extend along opposite sides of the reservoir (4) from open bottom ends (14) to two interior channels (18) in the manifold (15). The mouthpiece (19) promotes a favorable tactile response for the user. The cartridge assembly (1) is usable for a period of time until the nicotine composition has been consumed after which the cartridge is replaced and a new cartridge is inserted into the device (2). The mouthpiece (19) may be integral with the rest of the cartridge assembly (1).

Open bottom ends (14) extend to the air-let channels/conduits (12). The two interior channels (18) merge into an open top end (16) in the manifold (15), forming an inhalation opening present in the mouthpiece (19) where a user inhales the nicotine vapor. The two interior channels (18) allow air to pass in or out, such as vapor from the nicotine composition present in the reservoir. The two interior channels (18) will mix the nicotine aerosol efficiently when passing therethrough.

The wick may (11) extend into the reservoir (4) when the cartridge assembly (1) is inserted into the medical electronic nicotine delivery device (2) to facilitate transfer of nicotine from the reservoir (4) to the heater (32) within the housing (26) of the medical electronic nicotine delivery device (2) when the plug (10) is removed. The wick (11) is inserted and penetrates into interior volume (8) of reservoir (4) to contact the nicotine composition contained in the reservoir. a nicotine saturated fibrous material (9) may be disposed within interior volume (8) of reservoir (4) and the wick (11) is configured to contact the nicotine saturated fibrous material (9) in order to transfer nicotine from the fibrous material (9) to the heater (32) within the housing (26) and the heater (32) will heat up the nicotine composition so that it will be vaporized and inhaled by a person using said device and the person will get a craving relief from smoking. In one exemplary embodiment, the fibrous material (9) is made of a porous material such as thermoplastic polymer, for example a polyester. Further, examples are polyethylene terephthalate (PET), polybutylene terephthalate (PBT) or a mixture thereof or a mesh as well as stainless steel. It is important to note that any suitable material may be utilized.

The wick (11) may be made from any suitable material that is resistant to nicotine and has suitable temperature resistance. The wick (11) comprises, consists essentially of or consists of a porous material, such as a porous polymer, such as a thermoplastic polymer, preferably a thermoplastic polyester (for example, porous polyethylene terephthalate or PET). The wick (11) is preferably rigid. It is important to note that the wick (11) of the present invention should preferably not be made from cotton because such wick would collapse onto itself because it is not sufficiently rigid to withstand repeated insertion into and removal from the cartridge assembly (1). The wick may be porous by being formed from a fibrous material, such as a fibrous polymer.

The anti-reuse element (21) may include a plurality of springs 27, which are configured to be disrupted, changed, or damaged upon removal of the cartridge assembly (1) from the electronic nicotine delivery system (2). By introducing such anti-reuse feature, i.e. the spring being destroyed during withdrawal of cartridge assembly (1) from the housing (26) of the medical electronic nicotine delivery device (2), the risk of the cartridge assembly (1) being reused is minimized. Minimizing the risk of cartridge assembly (1) reuse, prevents refilled or otherwise altered cartridges to be used with the medical electronic nicotine delivery device (2).

The fibrous material (9) may be formed as one or more piece(s) substantially filling out the full interior volume (8) of reservoir (4). When the wick (11) engages or penetrates the fibrous material (9) it will start transporting the solution present within the reservoir (4) out of the reservoir (4). The wick (11) may be soaked or saturated with the nicotine composition. The fibrous material (9) may comprise one fibrous material unit.

Mouthpiece (19) extends in two opposite flaps (36) that will fit smoothly with a housing (26) of the electronic nicotine delivery device (2) (see FIG. 8). Flaps (36) provide a comparatively rigid connection between the cartridge assembly (1) and the housing (26) when the cartridge assembly (1) is fully inserted in the housing (26). Body assembly (3) of cartridge assembly (1) may be provided with a tamper protection device or an anti-counterfeit element (22). The anti-counterfeit element (22) may comprise a plurality of electrically conducting pads (23) (e.g. three pads) arranged at predetermined exterior positions on body assembly (3). It is important to note that the plurality of pads (23) are in a particular configuration; for example, a linear arrangement. In alternate approaches, a different number of pads (23) may be utilized in any suitable configuration that matches the electrical contact elements (24) on the inside of the housing (26) (see FIG. 11). When the cartridge assembly (1) is inserted into the housing (26), the three pads (23) make contact with electrical contact elements (24), which complete a circuit that measures resistance. Essentially, when the cartridge assembly (1) is moved relative to and within the housing (26) the anti-counterfeit element (22) is affected by matching electrical contact elements (24) on the inside wall of housing (26). Any simple circuit for measuring resistance may be utilized, for example, a Wheatstone bridge. If the resistance matches the preset resistance value programmed into the controller of the electronic nicotine delivery device (2), the cartridge assembly (1) is the proper one for the medical electronic nicotine delivery device (2) and the medical electronic nicotine delivery device (2) will work. If the resistance does not match, the cartridge assembly (1) is not the proper one for the medical electronic nicotine delivery device (2) and the medical electronic nicotine delivery device (2) will not work. It is important to note that the plurality of pads (23), for example three pads (23), may have equal resistance values, or different resistances values. What is measured is the total resistance when the circuit is completed by the insertion of the cartridge assembly (1) into the housing (26). Other electrical characteristics may be measured in place of, or in addition to, the resistance values, for example, capacitance. Once again there are well known simple circuits that may be utilized to measure capacitance as well as any number of other electrical parameters. The plurality of electrical contact elements (24) may be made from any suitable material and mounted to circuit board (25) by any suitable means. The choice of material is determined by the selected parameter to be measured which may also impact the means of attachment of the plurality of electrical contact elements (24) to the circuit board (25).

The cartridge assembly (1) and medical electronic nicotine delivery device (2) may comprise anti-reuse feature(s) as briefly set forth above. A plurality of springs (27) may be positioned in such a configuration/position that permits contact with plurality of electrical pads (28) (see FIG. 11) inside housing (26). In this position/configuration, it is possible to determine electrical properties, such as an electrical resistance, between the plurality of springs (27). A circuit board (25) within housing (26) contacts the plurality of springs (27) at electrical pads (28) when the cartridge assembly (1) is in position inside the housing (26) and determines preset electrical properties. More specifically, the plurality of springs (27) make contact with elements within the housing (26) in order to complete the circuit when the cartridge assembly (1) is inserted into housing (26). The controller within the medical electronic nicotine delivery device (2) determines that the circuit is complete by measuring a particular electrical parameter such as resistance; however, other parameters may be utilized. Upon removal of the cartridge assembly (1) from the housing (26), one or more of the plurality of springs (27) (e.g. one or both springs (27) when two springs are present) are bent out of the way by a member (37) of the housing (26) such that if re-inserted, the bent spring(s) (27) will no longer make contact with corresponding electrical pads (28) in the housing (26) and the circuit will not be complete. The member (37) that bends the one or more springs (27) may be a simple wedge-like protrusion that only bends the springs (27) upon removal of the cartridge assembly (1). Alternatively, one or more springs (27) may be broken off rather than simply bent; however, a piece of the spring (27) may fall into the housing (26) and become stuck therein. Preferably, the springs (27) may comprise any suitable conductive material that is pliable or bendable. In an embodiment where the springs (27) are designed to break rather than to bend, the springs (27) may be formed from a conductive but brittle material. In addition, in alternative exemplary embodiments, the springs (27) may be replaced with any suitable conductive material that forms a complete circuit when properly aligned, for example, a conductive foil that tears away when the cartridge assembly is removed from the housing (26).

The housing (26) may be arranged with an internal edge that will separate the springs (27) when the cartridge is removed from the housing (26). If the same cartridge assembly (1) is inserted again, the electrical pads (28) mounted on circuit board (25) will not contact the springs (27) correctly and the electrical properties as determined by the electronic circuit will not correspond to the stored data. As a result, the medical electronic nicotine delivery device (2) will not be enabled for proper use.

The cartridge assembly (1) and the different components thereof should be made from a material that is resistant against nicotine or salts thereof. Examples of materials include any kind of polymeric materials such as polyester, polyacrylonitrile (PAN) resin (Anobex™), cyclic olefin copolymer or High-Density polyethylene (HDPE). The cartridge assembly (1) as defined above may be sealed at the bottom end by the plug (10) and the body assembly (3) and the mouthpiece (19) to prevent environmental particles from entering into the cartridge assembly (1) during storage and transportation.

While the foregoing description and drawings represent exemplary embodiments of the present disclosure, it will be understood that various additions, modifications and substitutions may be made therein without departing from the spirit and scope and range of equivalents of the accompanying claims. In particular, it will be clear to those skilled in the art that the present invention may be embodied in other forms, structures, arrangements, proportions, sizes, and with other elements, materials, and components, without departing from the spirit or essential characteristics thereof. In addition, numerous variations in the methods/processes described herein may be made within the scope of the present disclosure.

### Operation

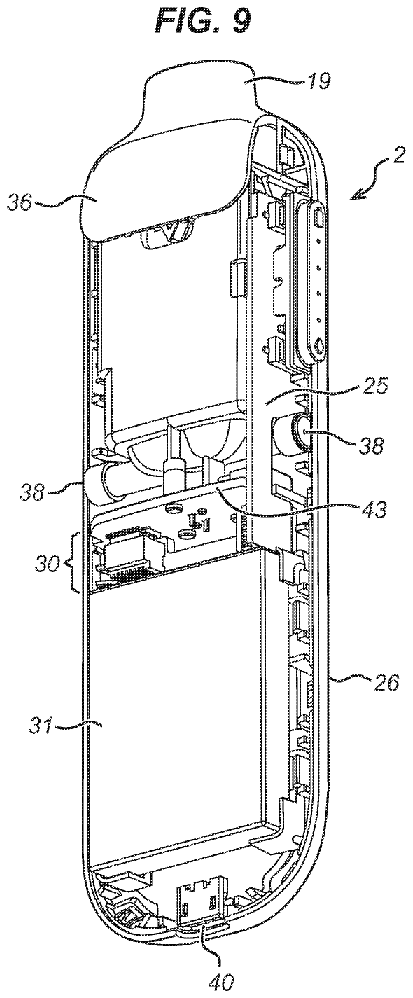

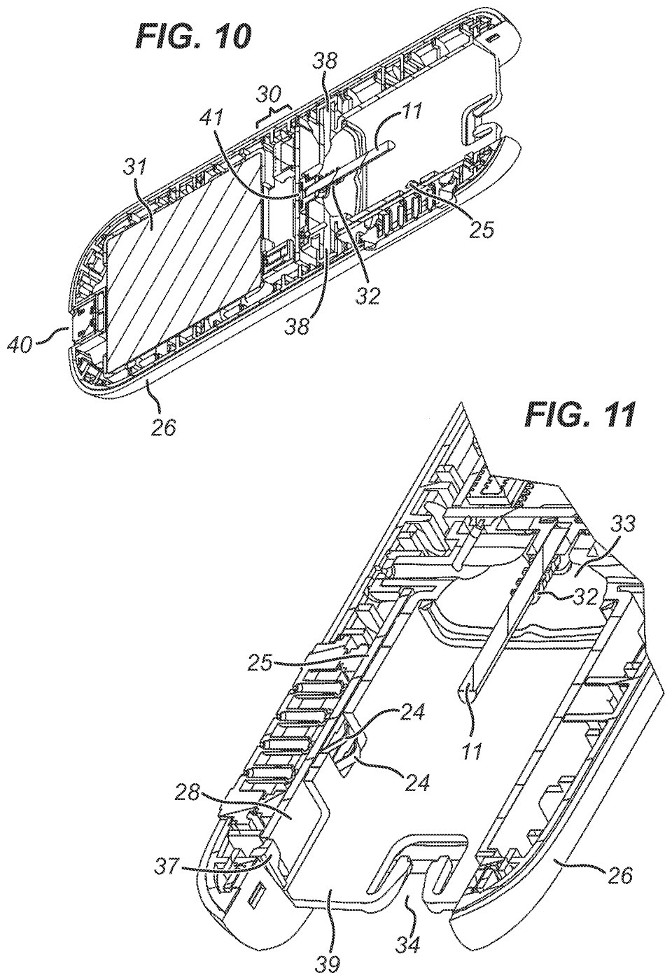

Referring back to FIGS. 1-11, the medical electronic nicotine delivery device (2) includes a power source (31) and a heater (32), preferably an electric heater or heating element, contained within a receptacle (39) (see FIG. 11). The receptacle (39) has at least one air inlet (38), see FIGS. 9 and 10, and provides holes, see FIG. 10, proximate the bottom wall of the body assembly (3), described in detail above, and proximate the heater (32). When assembled, the receptacle (39), heater (32), and cartridge assembly (1) cooperate to form a vaporization chamber (33) (see FIG. 11). The assembled electronic nicotine delivery system (2) also provides a predetermined airflow from the at least one air inlet (38) through the vaporization chamber (33), the conduits (12) and to the outlet (20) of the mouthpiece (19) to permit a user to inhale the nicotine aerosol formed therein. In addition, the assembled medical electronic nicotine delivery device (2) provides a liquid conduit from the reservoir (4) to the heater (32), preferably a wick (11), more preferably an elongate wick, as described above. The housing (26) may also provide for connectivity to an outside electrical source and/or data communication, such as a USB port (40), see FIG. 10, to supply and/or resupply the power source (31), preferably a rechargeable battery.

The power source (31) is sufficient to power the heater (32), a programmable controller (not shown), and any desired feedback to a user (e.g., light), external computer, or network. The programmable controller receives information from a pressure sensor (detecting inhalation by a user), and, possibly, other sensors (such as temperature sensors) to control the power delivered to the heater (32), and controls over temperature sensor(s), which can terminate power to the heater (32) to prevent undesirable and/or dangerous thermal events. The programmable controller may provide for data collection, storage and communication to the external computer. This may be communicated through a wireless connection. The power source (31) can be any appropriate portable power source (31) such as a primary or secondary battery or fuel cell.

The heater (32) is mounted on a holder (41) to isolate electrical resistance heater elements from other, thermally sensitive components of the medical electronic nicotine delivery device (2). The holder (41) may be formed from any suitable material with sufficient mechanical characteristics to support all elements attached thereto while providing an insulative function, i.e. low thermal conductivity. The heater (32) comprises at least one electrical resistance heater element contained in a heat diffusing material. The diffusion of the heat through the heat diffusing material generally evens out heat profile generated by the heating element(s) to prevent the formation of localized hot spots on the heater (32) surface.

As indicated above, the cartridge assembly (1) preferably includes a reservoir (4) containing a nicotine composition and a mouthpiece (19) for drawing a nicotine aerosol from the medical electronic nicotine delivery device (2). In addition, the assembled medical electronic nicotine delivery device (2) provides a liquid conduit from the reservoir (4) to the heater (32). In a preferred embodiment, the liquid conduit is a wick (11) (optionally an elongate wick) extending from the reservoir (4) to the heater (32). The wick (11) intimately contacts the heater (32) surface to enable the thermal energy to vaporize the nicotine composition transported thereto by the wick (11). As the nicotine composition is vaporized, the wick (11) transports additional nicotine composition to the heater (32) through capillarity.

The assembled medical electronic nicotine delivery device (2) also provides a vaporization chamber (33) proximate the heater (32). It is in the vaporization chamber (33) that the heater (32) vaporizes the nicotine composition transported by the wick (11) and in which the vaporized nicotine composition combines with outside air drawn in through one or more air inlets (38) to form a nicotine aerosol. The vaporization chamber (33) also communicates with the outlet (20) of the mouthpiece (19) via the interior channels (18) in the cartridge assembly (1) to permit a user to draw the nicotine aerosol into his or her mouth.

When a user draws air through outlet (20) from the mouthpiece (19), negative pressure causes air to flow into the assembled medical electronic nicotine delivery device (2) through one or more air inlets (38). In particular, air is withdrawn from the vaporization chamber (33) through the conduits (12) and the interior channels (18), lowering the air pressure in the vaporization chamber (33). The withdrawn air is replaced via air inlets (38) in the housing (26), proximate the vaporization chamber (33). The lowered air pressure in the vaporization chamber (33) is sensed by a pressure sensor disposed on the printed circuit board (43), outside of the vaporization chamber (33). The lower air pressure proximate the pressure sensor activates an operatively coupled switch with pressure sensor. This pressure sensor, in turn, activates the heater (32), which in turn heats the nicotine composition in contact therewith on the surface of the wick (11). The nicotine composition is vaporized and combined with air in the vaporization chamber (33), forming a nicotine aerosol. The nicotine aerosol is evacuated from the vaporization chamber (33) through the conduits (12) and interior channels (18) and delivered to the mouthpiece (19) and, ultimately, the user's mouth. As the nicotine composition is vaporized, additional solution is drawn from the reservoir (4) along wick (11) to heater (32). The volume of nicotine composition removed from reservoir (4) is replaced by air drawn through the at least one air inlet (38). In one exemplary embodiment, after a predetermined time (e.g., determined by the programmable controller), the power to the heater (32) is terminated, the vaporization chamber (33) cools, and no further nicotine aerosol is formed. The user will then stop drawing on the medical electronic nicotine delivery device (2), ending his/her “treatment”. Alternatively, the user may stop drawing on the medical electronic nicotine delivery device (2) prior to the predetermined time. In such a case, the pressure in the vaporization chamber (33) will return to atmospheric pressure and the pressure sensor will signal switch to terminate power to the heater (32).

In a final aspect the invention relates to a medical electronic nicotine delivery device (2), including a housing (26), a cartridge assembly (1) as defined above, an electrical module (30), a power source (31), a heater (32), and a vaporization chamber (33).

One skilled in the art will further appreciate that the embodiments may be used with many modifications of structure, arrangement, proportions, sizes, materials, and components and otherwise, used in the practice of the disclosure, which are particularly adapted to specific environments and operative requirements without departing from the principles described herein. The presently disclosed embodiments are therefore to be considered in all respects as illustrative and not restrictive. The appended claims should be construed broadly, to include other variants and embodiments of the disclosure, which may be made by those skilled in the art without departing from the scope and range of equivalents.

1. A nicotine containing cartridge assembly for use in a medical electronic nicotine delivery device comprising:
a body assembly having an interior surface defining an interior volume, the interior volume including a reservoir; wherein the reservoir has an interior volume, wherein the interior volume of the reservoir contains a composition comprising:
a. nicotine or a salt thereof in an amount of from about 2% w/w to about 6% w/w,
b. propylene glycol in an amount of from 50% w/w to about 65% w/w, and
c. water in an amount of from about 35% w/w to about 50% w/w.

2. The cartridge assembly of claim 1, wherein the reservoir is configured to receive a wick of the medical electronic nicotine delivery device, such that the wick contacts the composition.

3. The cartridge assembly of claim 2, wherein the reservoir comprises a port to allow the insertion of the wick therethrough.

4. The cartridge assembly of claim 2, wherein the wick comprises a porous polymer.

5. The cartridge assembly of claim 4, wherein the porous polymer is a porous polyester.

6. The cartridge assembly of claim 1, wherein the interior volume of the reservoir comprises a fibrous material or a sponge, wherein the fibrous material or sponge contains the composition.

7. The cartridge assembly according to claim 1, wherein the reservoir has a top wall, a bottom wall and side walls having an inner surface and an outer surface, the inner surface of side walls defining the interior volume of the reservoir.

8. The cartridge assembly of claim 7, additionally comprising two conduits formed between the outer surface of side walls of the reservoir and the interior surface of the body assembly, the conduits having open top ends 44 open bottom ends, and being configured for conducting aerosolized nicotine.

9. The cartridge assembly of claim 8, additionally comprising a manifold for holding and distributing aerosolized nicotine, the manifold positioned above the top wall of the reservoir, the manifold having an open top end and two open bottom ends, and at least one interior channel connecting the open top end with the open bottom ends, wherein the open bottom ends of the manifold are secured onto the open top ends of the conduits and are in fluid communication with the conduits.

10. The cartridge assembly of claim 9, further comprising a mouthpiece positioned above the manifold having an outlet in fluid communication with the at least one interior channel of the manifold, the mouthpiece configured for delivering aerosolized nicotine into the mouth of a user.

11. The cartridge assembly of claim 1, wherein the composition in reservoir comprises:
a. nicotine or a salt thereof in an amount of from about 2 to about 6% w/w,
b. propylene glycol in an amount of 60% w/w, and
c. water in an amount of 40% w/w.

12. The cartridge assembly of claim 1, further comprising an anti-counterfeit element mounted to the outer surface of the body assembly, the anti-counterfeit element configured to enable use of the cartridge assembly in the electronic nicotine delivery device.

13. The cartridge assembly of claim 12, wherein the anti-counterfeit element comprises a plurality of electrically conducting pads mounted on a substrate attached to the body assembly of the cartridge assembly, the plurality of electrically conducting pads configured to make electrical contact with corresponding electrical contact elements on a circuit board within a housing of the medical electronic nicotine delivery device, the plurality of electrically conducting pads being arranged in a predetermined pattern having predetermined electrical characteristics.

14. The cartridge assembly of claim 1, further comprising an anti-reuse element mounted to the outer surface of the body assembly, the anti-reuse element configured to preclude reuse of the cartridge assembly once removed from the medical electronic nicotine delivery device.

15. The cartridge assembly of claim 14, wherein the anti-reuse element comprises one or more springs configured to make electrical contact with a corresponding electrical pad mounted on circuit board within a housing of the medical electronic nicotine delivery device, the one or more springs being configured to be disrupted upon removal of the cartridge assembly from the housing.

16. A kit comprising at least one nicotine containing cartridge assembly of claim 1 and the medical electronic nicotine delivery device.

17. The kit of claim 16 comprising at least two cartridge assemblies.

18. A medical electronic nicotine delivery system comprising the cartridge assembly of claim 1 and the medical electronic nicotine delivery device.

19. The medical electronic nicotine delivery system of claim 18, wherein the medical electronic delivery device comprises a housing, an electrical module, a power supply, a heater, and a vaporization chamber.

20. Use of a nicotine-containing composition to reduce degradation of a component of a medical electronic nicotine delivery system during the use of the system, wherein the composition comprises:
nicotine or a salt thereof in an amount of from about 2% w/w to about 6% w/w, propylene glycol in an amount of from 50% w/w to about 65% w/w, and water in an amount of from about 35% w/w to about 50% w/w.
